(19) United States
(12) Patent Application Publication
(10) Pub. No.: US 2021/0140956 A1
(43) Pub. Date: May 13, 2021

(54) REFERENCE APTAMER SENSING ELEMENTS FOR EAB BIOSENSORS

(71) Applicant: Eccrine Systems, Inc., Cincinnati, OH (US)

(72) Inventors: Mikel Larson, Cincinnati, OH (US); Leila Safazadeh Haghighi, Cincinnati, OH (US); Jacob A Bertrand, Montgomery, OH (US); Florika Macazo, Cincinnati, OH (US)

(73) Assignee: Eccrine Systems, Inc., Cincinnati, OH (US)

(21) Appl. No.: 16/605,145

(22) PCT Filed: Nov. 16, 2018
(86) PCT No.: PCT/US18/61557

§ 371 Date: Oct. 14, 2019

Related U.S. Application Data
(60) Provisional application No. 62/587,829, filed on Nov. 17, 2017.

(51) Int. Cl. G01N 33/543; C12N 15/11

(52) U.S. Cl. CPC G01N 33/54386; C12N 2310/16; G01N 2458/30; C12N 15/111

(57) ABSTRACT

Electrochemical aptamer-based (EAB) biosensing devices are described that provide drift correction and calibration to EAB sensor measurements of biofluid analyte concentrations by disclosing reference sensors that are configured to not interact with a target analyte, but otherwise mirror the performance of active EAB sensors within the expected application parameters of the device. Such reference sensors are configured to allow comparisons with their companion active sensors to track aptamer sensing element dissociation from an electrode surface, temperature-induced effects, redox moiety dissociation, and/or the effects of surface fouling. Some embodiments include separate electrodes for active and reference aptamer sensing elements. Other embodiments include a single electrode for both active and reference aptamer sensing elements. Single electrode embodiments include two or more distinct redox moieties.

### CROSS-REFERENCE TO RELATED APPLICATIONS

The present application claims priority to PCT/US18/61557, filed Nov. 16, 2018, and U.S. Provisional Application No. 62/587,829, filed Nov. 17, 2017, and has specification that builds upon U.S. Provisional Application No. 62/523,835, filed Jun. 23, 2017, the disclosures of which are hereby incorporated herein by reference in their entirety.

### BACKGROUND OF THE INVENTION

Sweat contains many of the same biomarkers, chemicals, or solutes that are carried in blood and can provide significant information enabling one to diagnose illness, health status, exposure to toxins, performance, and other physiological attributes even in advance of any physical sign. Sweat itself, the action of sweating, and other parameters, attributes, solutes, or features on, near, or beneath the skin, or within interstitial fluid, also can be measured to further reveal physiological information. Recent progress in the development of wearable sweat sensing devices has been limited to high concentration analytes (μM to mM) sampled at high sweat rates (>1 nL/min/gland) found in, for example, athletic applications. However, progress will be much more challenging as wearable biosensing moves towards detection of large, low concentration analytes (nM to pM and lower).

In particular, many known sensor technologies for detecting biofluid solutes are ill-suited for use in wearable biofluid sensing, which requires sensors that permit continuous or extended use on a wearer's skin. This means that sensor modalities that require complex microfluidic manipulation, the addition of reagents, or the use of limited shelf-life components, such as antibodies, will not be sufficient for wearable sensing. What is needed is a stable, reliable, reagentless sensor that is sensitive to target analytes in biofluid, while providing the level of specificity necessary to produce high predictive values during the lifespan of the sensor. One solution to this problem is the use of electrochemical aptamer-based (“EAB”) sensor technology, such as the multiple-capture EAB biosensors (“MCAS”) disclosed in U.S. Pat. Nos. 7,803,542 and 8,003,374, or the docked aptamer EAB biosensors (DAS) disclosed in U.S. Provisional Application No. 62/523,835, filed Jun. 23, 2017, each of which is hereby incorporated by reference herein in its entirety.

Unfortunately, both types of EAB sensors can be vulnerable to errors caused by physical degradation of the individual aptamer sensing elements, changes in sensor responses that are due to fouling, or changes in environmental conditions, rather than changes in target analyte concentrations. Over time, aptamer sensing elements within an EAB sensor will physically degrade, meaning the sensing elements will become unattached to the electrode surface, or that parts of the sensing elements will disassociate from the sensing element structure. For example, analyte capture complexes can gradually detach from their respective docks, the docks themselves can detach from the electrode surface, or redox moieties can detach from the aptamer. Another source of error is non-specific binding to the aptamer sensing element or the electrode surface. Biofluids such as sweat, blood, saliva, or interstitial fluid contain numerous solutes, including large proteins. These can bind randomly to aptamer sensing elements or the electrode surface, altering or hindering sensor response to an analyte. Similarly, changes in external weather, internal temperature, and biofluid sample potential of hydrogen (pH) and salinity can affect the rate at which the sensors degrade and can affect the sensor response to target analyte concentrations. Others in the field of EAB sensing have posed solutions to similar problems, see, e.g., Li, H., et al., “Dual-reporter drift correction to enhance the performance of electrochemical aptamer-based sensors in whole blood,” J. Am. Chem. Soc., 10.1021/jacs.6b08671, 2016 (disclosing an EAB sensor with a pair of redox moieties attached to the aptamer, each having non-overlapping potential ranges, one of which moves in response to analyte capture and the other which serves as a reference); see also PCT/US18/58020, filed Oct. 29, 2018 (disclosing chronoamperometric EAB interrogation, which provides a signal that is independent of the number of aptamer sensing elements on the electrode surface).

Therefore, some embodiments of the disclosed invention include a reference EAB sensor to provide drift correction and calibration for a companion active EAB sensor. Other embodiments include reference sensor elements incorporated alongside active sensing elements within the same sensor to provide similar drift correction and calibration. Such devices and methods are the subject of the present disclosure.

### SUMMARY OF THE INVENTION

Electrochemical aptamer-based (EAB) biosensing devices are described that provide drift correction and calibration to EAB sensor measurements of biofluid analyte concentrations by disclosing reference sensors that are configured to not interact with a target analyte, but otherwise mirror the performance of active EAB sensors within the expected application parameters of the device. Such reference sensors are configured to allow comparisons with their companion active sensors to track aptamer sensing element dissociation from an electrode surface, temperature-induced effects, redox moiety dissociation, and/or the effects of surface fouling. Some embodiments include separate electrodes for active and reference aptamer sensing elements. Other embodiments include a single electrode for both active and reference aptamer sensing elements. Single electrode embodiments include two or more distinct redox moieties.

### BRIEF DESCRIPTION OF THE DRAWINGS

The present disclosure will be further appreciated in light of the following detailed descriptions and drawings in which:

FIGS. 1A and 1B are representations of a previously-disclosed MCAS aptamer sensing element.

FIGS. 1C and 1D represent a portion of an MCAS sensor of the disclosed invention that includes active and reference aptamer sensing elements.

FIGS. 2A and 2B are representations of a previously-disclosed DAS aptamer sensing element.

FIGS. 2C and 2D represent a portion of a DAS sensor of the disclosed invention that includes active and reference aptamer sensing elements.

FIGS. 3A and 3B depict an example embodiment of the disclosed invention, including an MCAS active sensor and a companion MCAS reference sensor respectively, in which multiple sensing elements are depicted interacting with target analytes.

FIG. 4 depicts an example embodiment of the disclosed invention, including at least a portion of a biofluid sensing device including at least one reference EAB sensor.

### DEFINITIONS

Before continuing with a detailed description of the exemplary embodiments, a variety of definitions should be made, these definitions gaining further appreciation and scope in the detailed description and embodiments of the present disclosure.

As used herein, “sweat” means a biofluid that is primarily sweat, such as eccrine or apocrine sweat, and may also include mixtures of biofluids such as sweat and blood, or sweat and interstitial fluid, so long as advective transport of the biofluid mixtures (e.g., flow) is primarily driven by sweat.

As used herein, “biofluid” may mean any human biofluid, including, without limitation, sweat, interstitial fluid, blood, plasma, serum, tears, and saliva.

“Biofluid sensor” means any type of sensor that measures a state, presence, flow rate, solute concentration, solute presence, in absolute, relative, trending, or other ways in a biofluid. Biofluid sensors can include, for example, potentiometric, amperometric, impedance, optical, mechanical, antibody, peptide, aptamer, or other means known by those skilled in the art of sensing or biosensing.

“Analyte” means a substance, molecule, ion, or other material that is measured by a biofluid sensing device.

“Measured” can imply an exact or precise quantitative measurement and can include broader meanings such as, for example, measuring a relative amount of change of something. Measured can also imply a binary or qualitative measurement, such as ‘yes’ or ‘no’ type measurements.

“Chronological assurance” means the sampling rate or sampling interval that assures measurement(s) of analytes in biofluid in terms of the rate at which measurements can be made of new biofluid analytes emerging from the body. Chronological assurance may also include a determination of the effect of sensor function, potential contamination with previously generated analytes, other fluids, or other measurement contamination sources for the measurement(s). Chronological assurance may have an offset for time delays in the body (e.g., a well-known 5- to 30-minute lag time between analytes in blood emerging in interstitial fluid), but the resulting sampling interval is independent of lag time, and furthermore, this lag time is inside the body, and therefore, for chronological assurance as defined above and interpreted herein, this lag time does not apply.

“EAB sensor” means an electrochemical aptamer-based biosensor that is configured with a plurality of aptamer sensing elements that, in the presence of a target analyte in a fluid sample, produce a signal indicating analyte capture, and which signal can be added to the signals of other such sensing elements, so that a signal threshold may be reached that indicates the presence or concentration of the target analyte. Such sensors can be in the forms disclosed in U.S. Pat. Nos. 7,803,542 and 8,003,374 (the “Multi-capture Aptamer Sensor” (MCAS)), or in U.S. Provisional Application No. 62/523,835 (the “Docked Aptamer Sensor” (DAS)).

“Analyte capture complex” means an aptamer, or other suitable molecules or complexes, such as proteins, polymers, molecularly imprinted polymers, polypeptides, and glycans, that experience a conformation change in the presence of a target analyte, and are capable of being used in an EAB sensor. Such molecules or complexes can be modified by the addition of one or more linker sections comprised of nucleotide bases.

“Aptamer sensing element” means an analyte capture complex that is functionalized to operate in conjunction with an electrode to detect the presence of a target analyte. Such functionalization may include tagging the aptamer with a redox moiety, or attaching thiol binding molecules, docking structures, or other components to the aptamer. Multiple aptamer sensing elements functionalized on an electrode comprise an EAB sensor.

“Reference EAB sensor” means a reference sensor that comprises aptamer sensing elements functionalized on an electrode base, where the aptamers do not interact with target analyte molecules, or have reduced interaction with target analyte molecules. A reference EAB sensor is configured to perform similarly to a comparable active EAB biosensor to facilitate calibration for one of more sources of drift or error.

“Reference aptamer sensing element” means an individual aptamer sensing element that is configured to have no or reduced interaction with target analyte molecules, but otherwise performs similarly to a comparable active aptamer sensing element. A plurality of reference aptamer sensing elements may be incorporated with active sensing elements to comprise an EAB sensor with built-in reference capabilities.

“Sensitivity” means the change in output of the sensor per unit change in the parameter being measured. The change may be constant over the range of the sensor (linear), or it may vary (nonlinear).

“Signal threshold” means the combined strength of signal-on indications produced by a plurality of aptamer sensing elements that indicates the presence of a target analyte.

### DETAILED DESCRIPTION OF THE INVENTION

One skilled in the art will recognize that the various embodiments may be practiced without one or more of the specific details described herein, or with other replacement and/or additional methods, materials, or components. In other instances, well-known structures, materials, or operations are not shown or described in detail herein to avoid obscuring aspects of various embodiments of the invention. Similarly, for purposes of explanation, specific numbers, materials, and configurations are set forth herein in order to provide a thorough understanding of the invention. Furthermore, it is understood that the various embodiments shown in the figures are illustrative representations and are not necessarily drawn to scale.

Reference throughout this specification to “one embodiment” or “an embodiment” means that a particular feature, structure, material, or characteristic described in connection with the embodiment is included in at least one embodiment of the invention, but does not denote that they are present in every embodiment. Thus, the appearances of the phrases “in an embodiment” or “in another embodiment” in various places throughout this specification are not necessarily referring to the same embodiment of the invention. Further, “a component” may be representative of one or more components and, thus, may be used herein to mean “at least one.”

Certain embodiments of the disclosed invention show sensors as simple individual elements. It is understood that many sensors require two or more electrodes, reference electrodes, or additional supporting technology or features which are not captured in the description herein. Sensors are preferably electrical in nature, but may also include optical, chemical, mechanical, or other known biosensing mechanisms. Sensors can be in duplicate, triplicate, or more, to provide improved data and readings. Sensors may be referred to by what the sensor is sensing, for example: a biofluid sensor; an impedance sensor; a sample volume sensor; a sample generation rate sensor; and a solute generation rate sensor. Certain embodiments of the disclosed invention show sub-components of what would be sensing devices with more sub-components needed for use of the device in various applications, which are obvious (such as a battery), and for purposes of brevity and focus on inventive aspects, such components are not explicitly shown in the diagrams or described in the embodiments of the disclosed invention. As a further example, many embodiments of the disclosed invention could benefit from mechanical or other means known to those skilled in wearable devices, patches, bandages, and other technologies or materials affixed to skin, to keep the devices or sub-components of the skin firmly affixed to skin or with pressure favoring constant contact with skin or conformal contact with even ridges or grooves in skin, and are included within the scope of the disclosed invention.

The detailed description of the present invention will be primarily, but not entirely, limited to devices, methods and sub-methods using wearable biofluid sensing devices. Therefore, although not described in detail here, other essential steps which are readily interpreted from or incorporated along with the present invention shall be included as part of the disclosed invention. The disclosure provides specific examples to portray inventive steps, but which will not necessarily cover all possible embodiments commonly known to those skilled in the art. For example, the specific invention will not necessarily include all obvious features needed for operation. Several specific, but non-limiting, examples can be provided as follows. The invention includes reference to the article in press for publication in the journal IEEE Transactions on Biomedical Engineering, titled “Adhesive RFID Sensor Patch for Monitoring of Sweat Electrolytes”; the article published in the journal AIP Biomicrofluidics, 9 031301 (2015), titled “The Microfluidics of the Eccrine Sweat Gland, Including Biomarker Partitioning, Transport, and Biosensing Implications”; as well as PCT/US16/36038, and U.S. Provisional Application No. 62/327,408, each of which is included herein by reference in their entirety. Techniques for concentrating a biofluid sample are disclosed in PCT/US16/58356, and U.S. Provisional Application No. 62/457,604, which are also hereby incorporated herein by reference in their entirety.

The embodiments described herein address a need to provide drift correction and calibration for electrochemical aptamer-based sensors through the use of reference sensors or reference sensing elements. For example, by including reference EAB sensors, a biofluid sensing device can compare the behavior of the reference sensor to that of the active sensor during the sensors' exposure to target analyte molecules. There are a number of data points obtainable through such a comparison. For example, both MCAS and DAS sensors can degrade over time through dissociation of components from the aptamer sensing elements or the sensing elements from the electrode. In addition, normal use of DAS sensors results in the loss of aptamer sensing elements, since analyte capture causes irreversible detachment of the aptamer from the electrode. A contemporaneous comparison of the reference EAB's signal to that of the active sensor can therefore reveal the number of functional sensor elements at the time of sensor use, so that signal strength can be more accurately correlated with analyte concentration. Such corrective inputs may take the form of electronic corrections to output signals, or may be applied via algorithm.

Similarly, non-specific binding, both to the aptamer sensing elements and to the electrode surface, can alter the signal produced by EAB sensors in the presence of target analyte molecules. Such fouling can cause steric hinderance or change an aptamer's secondary structure, flexibility, or other property. Large proteins can settle onto aptamer sensing elements, physically hindering target analyte interaction. All of these effects change the aptamer's reaction to target analyte molecules, and alter the signals that would result from such interaction. In addition, non-specific binding to the electrode surface can interfere with redox proximity to the electrode, hindering electrical response to target analyte, for example, by preventing the redox from coming close enough to the electrode to allow electron exchange.

As a specific example of a non-specific binding effect, pH variability, which reflects H+ ion concentrations in the biofluid, can alter sensing element behavior by providing different binding opportunities between the sensing elements and H+ ions. The number and location of H+ ions that bind to the sensing elements can significantly alter their folding characteristics, which again affects signal strength in the event of analyte binding. Biofluid salinity acts similarly to pH, in that different levels of ions in the biofluid present different probabilities of binding between the sensing elements and ions, which translates to altered signal output from the sensor. Together, these characteristics can significantly influence EAB sensor signals, and the reference capability disclosed herein will allow biofluid sensing devices to account for such influences and better isolate sensor signal due to analyte capture.

Another point of comparison between active and reference sensors is the effect of the biofluid sensing environment on EAB sensor signal response. Environmental factors such as outside weather and internal ambient temperature can alter the behavior of aptamer sensing elements, changing how they physically present themselves in relation to the electrode, both prior to and after analyte capture. For instance, the temperature of the sensor environment or of the biofluid itself can cause aptamer sensing elements to change their physical conformations. These conformation changes may bring the sensing elements' redox moieties closer to or further away from the sensor electrode, resulting in a change to the background signal produced by the sensor in the absence of target analyte molecules. Such factors can therefore alter the signal produced by the EAB sensor and affect how the signal is translated into an analyte concentration value.

With reference to FIG. 1A, a previously disclosed active MCAS aptamer sensing element is depicted. While the figure depicts, and the discussion focuses on, a single aptamer sensing element, EAB sensors described herein will include a large number (thousands, millions, or billions of individual sensing elements, having an upper limit of 10¹⁴/cm²) attached to the electrode. The aptamer sensing element 110 includes an analyte capture complex 112, which in turn is comprised of a randomized aptamer sequence 140 that is selected to interact with a target analyte molecule 160, and may include one or more linker nucleotide sections 142 (one is depicted). The analyte capture complex 112 has a first end covalently bonded to a dock 120, e.g., a sulfur molecule such as a thiol, which is in turn covalently bonded to a gold electrode base 130. The electrode 130 may be comprised of gold or another suitable conductive material. The sensing element further includes a redox moiety 150 that may be covalently bonded to a second end of the analyte capture complex 112 or bound to it by a linking section. In the absence of the target analyte, the aptamer 140 is in a first configuration, and the redox moiety 150 is in a first position relative to the electrode 130. When the device interrogates the sensing element using, e.g., square wave voltammetry (SWV), the sensing element produces a first electrical signal, eT_{A}.

With reference to FIG. 1B, the aptamer 140 is selected to interact with a target analyte 160, so that when the aptamer interacts with a target analyte molecule, the aptamer undergoes a conformation change that at least partially disrupts the first configuration, and forms a second configuration. The capture of the target analyte 160 accordingly moves the redox moiety 150 into a second position relative to the electrode 130. Now when the biofluid sensing device interrogates the sensing element, the sensing element produces a second electrical signal, eT_{B} that is distinguishable from the first electrical signal. After a recovery interval, the aptamer releases the target analyte, and the aptamer will return to the first configuration, which will produce the corresponding first electrical signal when the sensing element is interrogated.

With reference to FIG. 1C, an MCAS sensor that incorporates both active aptamer sensing elements 110 and reference aptamer sensing elements 105 is depicted. The active sensing elements 110 include the same components as depicted in FIG. 1A, including a first aptamer sequence 140 that is selected to interact with target analyte molecules. The reference sensing elements 105 include substantially similar elements to the active sensing elements 110, however, the second aptamer sequence 145 is configured not to interact with the target analyte 160, for example, through modifications that render it inactive.

In order to function as both an active and a reference sensor, the MCAS sensor described is configured so that the device can readily distinguish the active signals from the reference signals. In the depicted embodiment, the active sensing elements 110 are attached to a first electrode 130, while the reference sensing elements are attached to a second electrode 132. Other embodiments use a first redox moiety for the active sensing elements, and a second redox moiety for the reference sensing elements (not shown). Upon interrogation by the device, the active and reference sensing elements produce a first electrical signal, eT_{C}.

Referring now to FIG. 1D, when target analyte molecules 160 interact with the sensing elements, the active elements 110 capture the target, resulting in a conformation change that moves the redox moiety 150 closer to the first electrode 130. This relative movement produces a second signal, eT_{D}, upon interrogation of the electrode. Meanwhile, the reference elements 105 do not capture target analyte molecules, and therefore remain unmoved relative to the second electrode 132, producing a third electrical signal, eT_{E}. The third electrical signal is likely close to the first signal eT_{C}, but may reflect differences caused by changes in biofluid sample salinity, pH, temperature, or due to other factors, such as environmental change or sensing element degradation.

With reference to FIG. 2A, a previously disclosed active DAS aptamer sensing element is depicted. The aptamer sensing element 210 includes an analyte capture complex 212 and a molecular docking structure 220 immobilized on an electrode 230. The docking structure 220 may be attached to the electrode 230 by covalently bonding a first end to a thiol, which is, in turn, covalently bonded to the electrode. The docking structure 220 includes a nucleotide sequence that is selected to be complementary with a nucleotide sequence on the analyte capture complex 212, specifically, the dock is configured to pair with a first primer section 242. A redox chemical moiety 250 is immobilized on the unattached end of the dock 220, on the opposite end of the dock from the electrode 230. The dock 220 further includes two complementary nucleotide sequences 222, 224. In the initial arrangement, the analyte capture complex 212 is attached to a dock 220 that is attached to the electrode 230. When the dock is bound to the analyte capture complex, it is stiffened so that the redox moiety 250 is located at a maximum distance from the electrode 230, and creating a first signal prior to analyte capture, eT_{A}.

With reference to FIG. 2B, the active DAS is exposed to a biofluid sample containing a concentration of the target analyte 260. Upon interaction with the target analyte, the aptamer 240 interacts with the analyte 260 to capture the analyte, causing the second primer 244b to move into physical proximity to the first primer 242b. The physical proximity of the complementary primers causes the first primer to break free from the dock 220 and bind to the second primer 244b, allowing the complex to move away from the docking structure 220. Once the dock 220 is unbound from the first primer 242b, the dock becomes more flexible, and the complementary sections 222b, 224b bind together. The folding of dock 220 caused by the sections binding locks the attached redox moiety 250 in a position closer to the electrode 230, thereby producing a second signal, eT_{B}, upon interrogation.

With reference to FIG. 2C, a DAS sensor that incorporates both active aptamer sensing elements 210 and reference aptamer sensing elements 205 is depicted. The active aptamer sensing elements 210 include the same components as depicted in FIG. 2A, including a first aptamer sequence 240 that is selected to interact with target analyte molecules. In this embodiment, the active sensing element also includes a first redox moiety 250. The reference aptamer sensing elements 205 include substantially similar elements to the active sensing element 210, including the same complementary or primer regions, however, the second aptamer sequence 245 does not interact with the target analyte 260. As discussed above in relation to FIGS. 1C and 1D, the second aptamer 245 may be a modified version of the first aptamer, or may be a specially selected aptamer that behaves like the first aptamer without interacting with the target analyte. Further, in this embodiment, the reference sensing element also includes a second redox moiety 252, which produces a signal that is distinguishable from the first redox moiety 250. Other embodiments may instead use separate electrodes, as previously discussed in relation to FIGS. 1C & 1D, to produce distinguishable active and reference signals. Upon interrogation by the device, the active and reference sensing elements produce a first electrical signal, eT_{C}.

With reference to FIG. 2D, when target analyte molecules 260 interact with the sensing elements, the active elements 210 capture the target, causing the aptamer complex to break free and move away from the dock. The dock 220 then becomes flexible enough to allow the complementary sections bind together, which locks the redox moiety 250 closer to the electrode 230, producing a second signal eT_{D} upon interrogation. Meanwhile, the reference elements 205 do not capture the target analyte, leaving the second redox moiety 252 relatively unmoved relative to the electrode, producing a third electrical signal eT_{E} on interrogation. The third electrical signal is likely close to the first signal eT_{C}, but may reflect differences caused by changes in biofluid sample salinity, pH, temperature, or due to other factors, such as environmental change or sensing element degradation.

Tracking an EAB sensor's complement of functional sensing elements could prove especially useful for monitoring DAS function, since the normal use of the sensor causes the loss of analyte capture complexes. The loss of analyte capture complexes will necessarily reduce the amount of signal change available to the sensor when exposed to target analyte in the biofluid, and therefore affects the sensitivity of the sensor. A reference sensor could therefore calibrate the sensor by establishing a baseline signal that reflects the operational age of the active sensor (by accounting for time-based or use-based sensing element degradation), and changes due to biofluid characteristics. Such a baseline signal can then be compared to the active DAS signal to isolate the contribution of target analyte capture to the active DAS sensor signal. On subsequent uses, the baseline signal can also isolate the decrease in active sensor signal strength due to normal sensing element loss, i.e., analyte capture complexes that capture analytes and detach from their respective docks. If the background decay of both the reference and the sensing element are equivalent, then the difference of the two can be used to approximate the total accumulation of analyte over time.

Reference sensors or sensing elements may have a number of modifications with respect to their active sensor counterparts that allow the reference sensors to perform their desired calibration function. One such category of modifications includes the replacement or alteration of the randomized aptamer sequence that is selected to interact with the target analyte. For instance, at least a portion of the aptamer sequence used in the active sensing elements may be rearranged or randomized, so that the reference aptamer will not bind with the target analyte. Typically, aptamer sequences that interact strongly with a target analyte will have active binding sites that interact with the analyte interspersed along the sequence. One or more of these active binding sites can be disrupted by replacing the nucleotide base(s) at the active site with different bases, by moving the active site to another location on the aptamer, or by other suitable method. Alternatively, causing point mutations in portions of the aptamer not actively involved with binding the analyte may also disrupt the secondary structure enough to prevent binding with the analyte. Similarly, nucleotide bases may be replaced by non-native bases, or aptamers with different chirality, e.g., spiegelmers or left-handed ribonucleic acid (L-RNA) aptamers, may be used to reduce interaction with the analyte. In addition, entirely different aptamers may be used for the reference aptamer, for example, an aptamer selected to interact with a target unlikely to be present in the biofluid. Or the reference aptamer may be selected to have behavioral traits similar to the active aptamer without interacting with the target analyte, for example, aptamers having similar secondary structures to the active aptamer sequence. Such aptamers can be identified and selected through the use of isothermal titration calorimetry (“ITC”), nuclear magnetic resonance spectroscopy, x-ray crystallography, differential scanning calorimetry, or other suitable techniques which reveal the aptamer secondary structure.

Reference sensing elements or reference sensors may be configured to track specific sources of drift or error, or may be configured to track drift generally, which represents a composite of specific sources of error. For example, one source of error is dissociation of aptamer sensing elements from the electrode surface. Dissociation has many potential causes, including excessive temperatures, exposure to light and other radiation, oxidation, exposure to biofluid solutes, and other pathways known in the art. A simplified “dummy” reference sensor or sensing element may be configured to track aptamer dissociation by replacing the active aptamer with a non-nucleotide sequence, such as a simple carbon chain, or by bonding the redox moiety directly to a thiol or a docking structure without including an aptamer or aptamer substitute.

Another variable affecting EAB sensor error or drift is temperature. As a first order consideration, temperature affects aptamer conformation response to analyte capture. A temperature sensor may be used to measure the ambient temperature in the vicinity of the aptamer, and provide a benchmark that may be used with a look up table to calibrate sensor response at a given temperature. As a second order consideration, higher ambient and/or biofluid temperatures, e.g., above 70° C., also cause or accelerate aptamer sensing element dissociation from the electrode surface. To track temperature-induced dissociation, a reference sensor may be functionalized with elements known to have a specific decay rate due to temperature effects. For example, the electrode surface may be affixed with a SAM, molecule, or polymer with known temperature-induced decay rates, or an aptamer sensing element, or dummy element, may be attached to redox moiety having a known temperature-induced dissociation profile. This temperature reference sensor would provide a temperature hysteresis correction factor for the active sensor. A reference may also provide a measure of the remaining lifetime of the active sensor or sensing elements by facilitating an estimate of remaining active sensing elements on a sensor.

Another source of error is dissociation of the redox moiety from the aptamer sensing element. Some embodiments accordingly will track such error by using a plurality of different redox moieties on active sensor elements or companion reference sensing elements. Each type of redox moiety would have a distinct electrochemical and/or chemical behavior profile. For example, the redox moieties can vary based on the numbers of exchanged electrons, reversibility of redox reaction, reaction speed, pH dependence, protonation constant, redox equilibrium constant, redox potential, susceptibility to electrochemically induced degradation, oxidation, relaxation mechanism, fluorescence, hydrophilicity, and amphiphilicity. The changes in signal responses between the redox moieties can provide corrective inputs as to sensor activity relevant to analyte concentration, or can provide inputs tracking sensor lifetime.

Another specific source of drift is the tendency of EABs to experience surface fouling due to solutes settling on, or bonding to, the aptamer sensing elements, the self-assembled monolayer, or the electrode surface. In embodiments configured to track surface fouling error, a filter or selectively permeable membrane may be placed upstream of a reference sensor to isolate the influence of larger proteins and other solutes on sensor response. Such a reference sensor would see less change due to large molecule non-specific binding, or aptamer and electrode surface fouling, which is compared to readings from unfiltered reference or active sensors. Alternatively, a plurality of depletion surfaces functionalized to covalently bond potentially interfering solutes can be placed upstream of reference or active sensors. Like filters, these depletion surfaces remove potentially fouling solutes from the biofluid, allowing for measurement and comparison to sensors measuring untreated biofluid. Some embodiments may include depletion zones between two active or reference sensors to compare measurements with and without fouling species present.

In addition to the proactive reference sensors and sensor elements described, some embodiments may be configured as passive reference sensors. One such passive reference sensor uses fluorescent tags that can be read to determine the amount of sensor dissociation over time. For example, an EAB sensor includes a plurality of reference aptamer sensing elements that have a fluorescent tag affixed to their redox moieties, to their docking structures, or elsewhere. As the EAB sensor degrades over time, the amount of fluorescence remaining on the EAB sensor is measured, e.g., with an optical sensor such as a photodiode, and the degree of dissociation determined.

With reference to FIGS. 3A and 3B, another embodiment of the disclosed invention is depicted, wherein a biofluid sensing device includes separate active and reference EAB sensors. For example, FIG. 3A depicts an active EAB sensor 320a, containing only, or substantially only, active aptamer sensing elements 322, 324, 326, 328, 332, 334. The active sensing elements include aptamer sequences 340 that are configured to interact with target analyte molecules 360. As shown, at a time after exposure to a concentration of target analyte in a biofluid sample, a number of active sensing elements 322, 326, 332 will capture analyte molecules 360, and some sensing elements 324, 328, 332 will not capture analytes. Further, due to the characteristics of the biofluid sample, such as pH, salinity, temperature, etc., some sensing elements will fold in various ways that move their respective redox moieties closer to, or further from, the electrode 330a. In addition, some sensing elements 328 may see degradation over time, resulting in movement of their respective redox moieties relative to the electrode 330a. Any of these scenarios can alter the total signal produced by the active sensor 320a, affecting the device's interpretation of analyte concentration.

FIG. 3B depicts a reference EAB sensor 320b, that is configured to be a companion sensor to the active sensor 320a. The reference sensor 320b contains reference aptamer sensing elements 321, 323, 325, 327, 329, 331, and includes aptamer sequences 345 that are configured to behave substantially similarly to the active sequences 340, except the reference sequences 345 will not interact with target analyte molecules 360. Upon exposure to a biofluid sample that is similar to that seen by the active sensor 320a, the reference sensing elements 321, 323, 325, 327, 329, 331 will not capture analyte molecules 360, but otherwise will exhibit similar probability rates of folding permutations and component degradation as the active aptamer sensing elements 322, 324, 326, 328, 332, 334 of FIG. 3A. Because of this similar behavior, the reference sensor signal can be compared to the active sensor signal, allowing the biofluid sensing device to account for background noise due to biofluid sample characteristics and degradation when interpreting the active sensor's measure of analyte concentration.

With reference to FIG. 4, a cross-sectional view of at least a portion of a biofluid sensing device employing reference EAB biosensors is depicted. The device 400 includes a water-impermeable substrate 410, a protective covering 412, a microfluidic channel 480, an inlet 482 and a sweat collector (not shown) to introduce a sweat sample into the device. The channel 480 is configured to concentrate a sweat sample relative to a target analyte, and includes an optional pre-concentration filter 492, a selectively-permeable concentrator membrane 490 and a concentrator pump 494. When a sweat sample enters the channel through the inlet, it moves in the direction of the arrow 16, where it encounters the pre-filter. The filter removes solutes from the sweat sample based on size, electrical charge, or chemical property, or removes proteases or other solutes that may interfere with the device measurements. Once through the filter, the sweat sample is concentrated relative to the target analyte by the concentrator membrane 490, which could be a dialysis membrane, or other material that at least allows the passage of water and inorganic solutes, but prevents passage of the target analyte. The pump 494 is constructed of a material suitable for drawing water out of the channel through the membrane.

As the sweat sample moves through the channel, it becomes increasingly concentrated, and interacts with at least one active EAB sensor 422, 424 and at least one reference EAB sensor 421, 423. In an alternate embodiment, instead of using separate active and reference EAB sensors, some devices will have one or more EAB sensors that include both active and reference aptamer sensing elements (not shown). Some embodiments also include one or more secondary sensors (not shown), which are one of the following: a micro-thermal flow rate sensor, one or more ISEs for measuring electrolytes (H⁺, Na⁺, Cl⁻, K⁺ Mg²⁺, etc.), a sweat conductivity sensor, a temperature sensor, or other sensor. Some embodiments also include a sweat stimulant gel 440 composed of sweat stimulant such as carbachol or pilocarpine, and agar, and an iontophoresis electrode 450. The electrode 450 can also be used to measure skin impedance or galvanic skin response (“GSR”), which indicates sweat onset or sweat cessation timing. In use, such a device 400 takes measurements produced by the active EAB sensors 422, 424, and compares them to measurements from the reference EAB sensors 421, 423, allowing signal output due to captured analyte molecules to be isolated from signal caused by other factors.

While several exemplary embodiments have been described herein, it is anticipated that other types configurations may also be used. Various modifications, alterations, and adaptations to the embodiments described herein may occur to persons skilled in the art with attainment of at least some of the advantages. The disclosed embodiments are therefore intended to include all such modifications, alterations, and adaptations without departing from the scope of the embodiments as set forth herein.

This has been a description of the disclosed invention along with a preferred method of practicing the disclosed invention, however the invention itself should only be defined by the appended claims.

1. A device, comprising:
a plurality of active aptamer sensing elements, each active aptamer sensing element including a first nucleotide sequence configured to interact with a target analyte, an active redox moiety, and a dock;
a plurality of reference aptamer sensing elements, each reference aptamer sensing element including a second nucleotide sequence, a reference redox moiety, and a dock, the second nucleotide sequence being configured to not interact with the target analyte;
an active electrode having the plurality of active aptamer sensing elements attached thereto, wherein the electrode is capable of detecting a first signal from the active redox moiety moieties; and
a reference electrode having the plurality of reference aptamer sensing elements attached thereto, wherein the electrode is configured to detect a second signal from the reference redox moieties.

2. The device of claim 1, wherein the active electrode and the reference electrode are configured as separate sensors.

3. The device of claim 1, further comprising one or more of the following:
a sensor configured to measure a potential of hydrogen of the biofluid;
a sensor configured to measure a concentration of Na+ in the biofluid; and
a sensor configured to measure a temperature in the vicinity of the plurality of active aptamer sensing elements and the plurality of reference aptamer sensing elements.

4. The device of claim 1, further comprising,
a microfluidic channel in fluidic communication with a source of biofluid, the active electrode, and the reference electrode, wherein the source of biofluid is located upstream of the active electrode and reference electrode; and
a selectively permeable membrane located between the source of biofluid and the reference electrode.

5. The device of claim 1, further comprising,
a microfluidic channel in fluidic communication with a source of biofluid, the active electrode, and the reference electrode, wherein the source of biofluid is located upstream of the active electrode and reference electrode; and
a depletion zone for removing solutes, wherein the depletion zone is located between the source of biofluid and the reference electrode.

6. The device of claim 1, wherein the second nucleotide sequence is the first nucleotide sequence having one of the following modifications: a base change at one or more active binding sites, one or more point mutations, and one or more bases replaced with a non-native base.

7. The device of claim 1, wherein the second nucleotide sequence is one of the following: a spigelmer, and a left-handed ribonucleic acid.

8. The device of claim 1, further comprising a sensor for measuring fluorescence, and wherein the plurality of reference aptamer sensing elements each further comprises a fluorescent tag.

9. The device of claim 1, wherein the plurality of reference aptamer sensing elements each further includes a temperature-induced dissociation profile.

10. A device, comprising:
a plurality of active aptamer sensing elements, each active aptamer sensing element including a first nucleotide sequence configured to interact with a target analyte, a first redox moiety configured to produce a first redox signal, and a dock;
a plurality of reference aptamer sensing elements, each reference aptamer sensing element including a second nucleotide sequence configured to not interact with the target analyte, a second redox moiety configured to produce a second redox signal that is distinguishable from the first redox signal, and a dock; and
an electrode having the plurality of active aptamer sensing elements and the plurality of reference aptamer sensing elements attached thereto, wherein the electrode is configured to detect signals from the first redox moieties and the second redox moieties.

11. The device of claim 10, wherein each of the plurality of reference aptamer sensing elements further comprises one or more of the following: a third redox moiety, a fourth redox moiety, and a fifth redox moiety; and
wherein the electrode is configured to detect signals from one or more of the third redox moiety, the fourth redox moiety, and the fifth redox moiety.

12. The device of claim 10, further comprising one or more of the following:
a sensor configured to measure a potential of hydrogen of the biofluid;
a sensor configured to measure a concentration of Na+ in the biofluid; and
a sensor configured to measure a temperature in the vicinity of the plurality of active aptamer sensing elements and the plurality of reference aptamer sensing elements.

13. The device of claim 10, further comprising,
a microfluidic channel in fluidic communication with a source of biofluid and the electrode, wherein the source of biofluid is located upstream of the electrode; and
a selectively permeable membrane located between the source of biofluid and the electrode.

14. The device of claim 10, further comprising,
a microfluidic channel in fluidic communication with a source of biofluid and the electrode, wherein the source of biofluid is located upstream of the electrode; and
a depletion zone for removing solutes, wherein the depletion zone is located between the source of biofluid and the electrode.

15. The device of claim 10, further comprising a sensor for measuring fluorescence, and wherein the plurality of reference aptamer sensing elements each further comprises a fluorescent tag.

16. A device, comprising:
a plurality of active aptamer sensing elements, each active aptamer sensing element including a first nucleotide sequence configured to interact with a target analyte, and a redox moiety;
a plurality of reference aptamer sensing elements, each reference aptamer sensing element including a second nucleotide sequence, and a redox moiety, the second nucleotide sequence being configured to not interact with the target analyte;
an active electrode having the plurality of active aptamer sensing elements attached thereto, wherein the electrode is capable of detecting a first signal from the active aptamer sensing element redox moieties; and
a reference electrode having the plurality of reference aptamer sensing elements attached thereto, wherein the electrode is capable of detecting a second signal from the reference aptamer sensing element redox moieties.

17. The device of claim 16, wherein the device compares the first signal with the second signal to track one or more of: aptamer sensing element dissociation from an electrode surface, temperature-induced effects, redox moiety dissociation, and surface fouling.
